(19) United States
(12) Patent Application Publication
(10) Pub. No.: US 2022/0280948 A1
(43) Pub. Date: Sep. 8, 2022

(54) BIOLOGICAL MATERIAL PURIFICATION DEVICE AND PROCEDURE USING MAGNETIC BEADS

(71) Applicant: TODARO ROBOTICS INC., San Diego, CA (US)

(72) Inventor: Thomas Todaro, San Diego, CA (US)

(21) Appl. No.: 17/653,298

(22) Filed: Mar. 3, 2022

Related U.S. Application Data
(60) Provisional application No. 63/156,092, filed on Mar. 3, 2021.

(51) Int. Cl. B01L 9/00

(52) U.S. Cl. CPC B01L 9/523; B01L 2200/0652; B01L 2200/026; G01N 2035/00574

(57) ABSTRACT

An automated workstation configured for magnetic bead nucleotide purification may be provided with a bead mover device, a working nest and a plate hotel. In some embodiments, the bead mover device includes a housing, a magnetic head assembly having an array of downwardly extending elongated magnets and a leadscrew located within the housing and configured to move the magnetic head assembly along a vertical axis. The working nest may be located on the frontside of the bead mover housing below the magnetic head assembly, and the plate hotel may be located on the backside of the housing and configured to hold a plurality of fixtures when not in use on the working nest. Methods of use are also disclosed.

### CROSS REFERENCE TO RELATED APPLICATIONS

This application claims the benefit of U.S. Provisional Application No. 63/156,092 filed Mar. 3, 2021, which is herein incorporated by reference in its entirety.

### FIELD

The present disclosure generally deals with automated machine systems, devices and procedures for the concentration, purification and/or isolation of biological material, such as nucleotides including DNA/RNA, proteins and cells. A practitioner of the genetic sciences will recognize that such a machine system is applicable and common in a variety of research activities and associated markets, including but not limited to biobanking/human genetics, human leucocyte antigens (HLA) typing, virus and bacteria detection.

The present disclosure particularly concerns an improvement to the device and the process of concentrating, purifying and/or isolating nucleotides using magnetic beads. The improved device and procedure of the present disclosure eliminate the time-consuming pipetting of individual microplate wells which require expensive disposable pipettor tips. The innovative device and procedure instead invert an existing geometry so that all the microplate wells (typically 96 in number) may instead be washed with, eluted into and/or entered with any of various fluid solutions and reagents required in parallel, and efficiently, all at the same time.

### BACKGROUND

### Existing Purification/Concentration Devices

Automated machine systems, devices and apparatuses for the concentration, purification an isolation of nucleotides including DNA/RNA, proteins and cells exist circa 2020. Certain of these apparatus, and associated methods of use, that have greater and faster throughput use a technique involving magnetic beads.

In the prior art technique, the purification processing is often performed in the wells of an industry-standard 96-well plate, or microplate. Samples and reagents are added to the wells of the microplates prior to processing. Magnetic beads coated with a material to which those nucleotides that are desired to be concentrated adhere are placed in the wells. Each well and its contained coated magnetic beads are then exposed to a distinct and identified unconcentrated solution of these nucleotides that is desired to be concentrated. The nucleotides adhere to the material, thus coating the magnetic beads.

Subsequently in the standard process for performing “magnetic bead purification”, a magnet is placed underneath a microplate. These magnets hold the beads (already within the microplate wells) to the bottoms of the microplate wells so that a pipettor can pipette around and over them, and wash them, while the beads stay held in place.

In detail, these steps of the standard process are automated in a machine process as follows. Step 1 is cell lysis. Cells in the microplate are lysed chemically, and lysis is enhanced in the mixture by mixing using the plastic tip cones of rods, one rod descending vertically (along the “Z” axis) into each well of the microplate and then, after physically mixing the contents of the well, ascending from the well of the microplate. Magnetic rods are used to collect magnetic particles on the tip cones.

Step 2 is when magnetic particles are collected from separate wells and brought to and deposited into sample wells for binding.

Step 3 is when magnetic particles are collected onto the tip cones. Magnetic rod speeds in the “Z” axis are normally easily adjusted by the software controlling the automated apparatus.

Step 4 is when target molecules—for example DNA, RNA, proteins or cells—are bound to the magnetic particles. Note that it is the particles that are being moved through the process, and not the liquids. For example, DNA becomes bound to the target particles and is thereafter moved along with the magnetic particles to which it is bound.

Step 5 is the washing of the target molecules. Dirty washing liquid is left behind. If necessary, any number of repetitive washing steps may be performed, and three (3) or more washings are normally preformed in sample purification.

This simple step 5 has severe disadvantages and consequences that will be seen to be effectively dealt with by the present disclosure. First it takes a long time to run the multiple washings using a pipettor. To avoid cross contamination, a large number of expensive disposable pipettor tips must typically be, and are, used in the process (it being impermissible to contaminate one well with the contents of another). The wells of a standard microplate may prove too small for effective washing of the (coated magnetic beads binding nucleotide) particles within the well by process of pipetting, and special “deep well” microplates may be required or desired, again significantly adding to process cost. If any special microplates have a different vertical dimension than does a standard microplate, then the Z-axis control of any apparatus or system moving the microplates (and their contents) will have to be correctly adjusted for this changed dimension.

Step 6 is the eluting of target molecules. Purified target molecules are released onto a small end volume. The sample is now ready for downstream application(s).

### SPECIFIC EXAMPLES OF AUTOMATED DEVICES PERFORMING “MAGNETIC BEAD PURIFICATION” OF NUCLEOTIDES

One example circa 2020 of an automated device performing “magnetic bead purification” of nucleotides is the KingFisher device from Thermo Fisher. This device combines an up and down motion of the magnets capturing beads that initially have nucleotides or proteins upon their surfaces, and that are present within the wells of a microplate. Many microplates are mounted circumferentially on a turntable. This turntable is pre-loaded and rotated to bring the microplates under a magnetic head as needed, thereby increasing throughput.

Since about 2019, and at the time of the filing of this application, there is a video of this process on the Internet entitled “KingFisher Flex System [by] Thermo Fisher Scientific” appearing at YouTube.com.

A similar product available from Perkin Elmer is called the “Chemagic”. Since about 2019, and at the time of the filing of this application, there is also a video of the Chemagic process appearing on the Internet as “Chemagic 360—High Quality DNA/RNA Isolation”, again at YouTube.com.

Both these prior art devices show the washing of the coated magnetic beads binding nucleotides that are present within the plate wells of a microplate by a process of repetitive washing with, and from, a pipettor. As previously explained, this is not optimal. The present disclosure will be seen to deal with this very process step.

Insofar as is possible, as well as speeding and improving the “magnetic bead purification” of nucleotides particularly in the washing step, a new device that was simpler with a much lower cost and price would be desirable. Such a simpler new device could desirably be capable of being set up on a bench and used manually. Because of its compact size, such a device could desirably be placed onto the deck of an automated pipetting system. Instead of proprietary plastic tips of a manufacturer such as Thermo-Fisher or Perkin Elmer, such a device could desirably use commonly sold, low-cost, PCR plates as the plastic covering for the magnetic tips.

Such a new device could be both simple to operate, and easy to maintain and repair.

Notably, circa 2020-2021 at the time of the filing of this patent application, both aforementioned devices and automated machine systems of Thermo Fisher and Perkin Elmer are severely back-ordered due to the coronavirus pandemic. These devices are used for PCR COVID-19 tests.

The “magnetic bead purification” of nucleotides is proven effective, and cost effective, and the “magnetic bead purification” of nucleotides is in no way repudiated by the present disclosure. The present disclosure should rather be considered an improvement to the prior art automated devices and systems, and the machine methods, for the “magnetic bead purification” of nucleotides.

### SUMMARY OF THE DISCLOSURE

As explained, a magnetic bead assay of the prior art permitted the magnetic bead purification of DNA and RNA (nucleotides). The standard process for performing this type of purification involved placing a magnet underneath a microplate. These magnets held the magnetic beads to the bottom of the plate's well so that a pipettor could pipette over them and around them, serving to wash them while the magnetic beads were magnetically held securely in place. Also as just explained in the BACKGROUND section, there are many disadvantages to this method, including the long time that it takes to run the washing step of the machine process using a pipettor, and the large number of expensive disposable pipettor tips that are used in the process.

The present disclosure contemplates hanging magnets downwards above (and not below) wells of the microplate—a “magnetic head”—and dipping these magnets downwards into the wells of the microplate so that the magnetic beads stick to the inserted magnets, and so that by subsequent upwards extraction of the magnets from the wells the beads and anything bound in or to their surfaces are moved into free space over the microplate. In this position the “magnetic head” with captured beads can be laterally moved relative to the microplate from which the magnetic beads were extracted, and even into position over the tops of still other microplates. In position over any microplate the “magnetic head” and its captured magnetic beads can again be moved up and down, meaning in and out of various reagents for washing and recovery.

Thus, the magnetic head and its capture magnetic beads (with nucleotides upon their surfaces) may be, in the raised position, moved laterally over the tops of various microplates containing various solutions, or, equivalently, the raising and lowering magnetic head may stay in one lateral position, and successive microplates with reagents in their wells can be laterally moved under the raised magnetic head. It is generally easier to have a stationary single site controlling the movement of the magnetic head, and to move new microplates with new reagents in their wells to this site than to move the magnetic read head with its magnetically held beads to positions atop various microplates. Witness the prior art where microplates were moved upon a turntable, albeit for a different purpose. In some embodiments of the present disclosure, a laterally stationary station where the “magnetic head” moves in the “Z” axis is provided, although a practitioner of the mechanical arts will recognize that two things may be mechanically moved relative to each other in many different ways.

By this simple expedient of moving the beads on magnets moved vertically from positions located above the beads in their wells as opposed to using magnets positioned below the wells (1) time is saved, and (2) any need for extensive pipettor tip usage is greatly reduced or eliminated.

The primary purpose for moving new microplates under the magnetic beads (with nucleotides on their surfaces) extracted upwards from their original wells, and then plunging the magnetic beads into the new wells of a new microplate, and repeating this process, is of course to wash, and to later elute, all the magnetic beads in parallel. These steps (of magnetic bead purification) can now be done efficiently and quickly in parallel on all magnetic beads. In fact, it is so easy and inexpensive to add an extra step—say, for example, an extra washing step of which there are normally three (3)—it may be contemplated that the magnetic bead purification process may be conducted to better effect, and/or modified to add new reagents in new steps yet to be determined.

An automated device and system, in accordance with the modified “magnetic bead purification” machine process, in some embodiments utilize a new (circa 2020) magnetic head that has been recently released as a product from a company called V&P Scientific, 9823 Pacific Heights Blvd T, San Diego, Calif. 92121. The operation of this magnetic head may be viewed circa 2020 in video entitled “Automate Your Magnetic Bead Wash with the VP 407AM-N1-R” appearing at YouTube.com.

Although this magnetic head is new, it is contemplated to be used in the washing step of industry-standard magnetic bead purification of DNA and RNA (nucleotides) quite as before. For example, the above video shows the use of the new magnetic head attached to an Opentrons pipettor. This is a very low-end device that is not usable for most labs. The new magnetic head is being sold as a stand-alone product as of the date of the filing of this application.

An automated, machine-performed, magnetic bead purification of DNA and RNA (nucleotides) in accordance with the present disclosure contemplates mounting a magnetic head—such as the new one from V&P Scientific just discussed—to a Z-axis motor and bracket. This motor and bracket simply serve to move the (magnetic) head up and down, into and out of, the ninety-six (96) wells of a standard microplate.

The microplates will contain different solutions needed for nucleotide purification. The plates will be moved in and out of the device nest, and the head will move down-hanging magnets up and down as needed into and out the wells of the microplates. Z axis operation will be controlled remotely by a computer. This device can sit on a bench and be operated manually or be integrated into a robot system on the deck of a pipetting device. The magnetic bead nucleotide purification device and system so improved is simple, and of low cost. It is capable of being set up on a bench and used manually. It has a compact size and is suitably placed onto the deck of an automated pipetting system. The improved device and system use no proprietary plastic pipettor tips of any manufacturer but instead use only commonly sold, low cost, PCR microplates as the plastic covering for the magnetic tips.

The improved device is simple to operate, and easy to maintain and repair.

According to aspects of the disclosure, in some embodiments an automated workstation configured for magnetic bead nucleotide purification includes a bead mover device, a working nest and a plate hotel. The bead mover device may include a housing, a magnetic head assembly having an array of downwardly extending elongated magnets, and a leadscrew located within the housing and configured to move the magnetic head assembly along a vertical axis. The working nest may be located on a frontside of the housing below the magnetic head assembly, and the plate hotel may be located on a backside of the housing. In some embodiments, the plate hotel is configured to hold a plurality of fixtures when they are not in use on the working nest.

In some embodiments, a workstation further includes a computer processor configured to control the leadscrew in moving the magnetic head along the vertical axis. The plate hotel may include a plurality of shelves attached to the backside of the bead mover device housing. In some embodiments, the plate hotel includes at least 6 shelves and in other embodiments it includes at least 8 shelves. In some embodiments, the shelves have an orientation that is parallel to the orientation of the working nest, and in other embodiments the shelves have an orientation that is perpendicular to the orientation of the working nest.

In some embodiments, the array of downwardly extending elongated magnets is configured to be removably covered by cover plate. The cover plate may be an industry standard ninety-six well microplate. The workstation may further include a cover plate loading manifold configured to removably receive the cover plate, be temporarily stored in the plate hotel, and transferred to the working nest to apply the cover plate to the array of magnets before use.

In some embodiments, the workstation further includes a cover plate ejection manifold configured to be temporarily stored in the plate hotel, transferred to the working nest, and receive the cover plate from the array of magnets after use. The magnetic head assembly may be provided with a pair of toggles on opposite sides of the magnetic head assembly configured to automatically eject the cover plate when the magnetic head assembly is lowered by the leadscrew onto the cover plate ejection manifold.

In some embodiments, the workstation further includes a base to which a bottom end of the bead mover device housing is attached. The working nest may include a lower frame provided with elongated slots that allow it to be adjusted and secured in an x-direction relative to the base and the bead mover device, and the working nest may include an upper frame also provided with elongated slots allowing it to be moved independently from the lower frame, adjusted and secured in a y-direction. This arrangement allows the working nest and therefore plates and fixtures that are alternately placed upon the working nest to be accurately registered relative to the array of downwardly extending magnets.

In some embodiments, the workstation further includes a base to which a bottom end of the bead mover device housing is attached. The base may be no larger than 15 inches wide and 15 inches deep, and the bead mover device, the working nest and the plate hotel may all fit within a vertical projection of the base.

According to aspects of the disclosure, a method of purifying biological material using magnetic beads may include providing an automated workstation. The workstation may include a bead mover device, a working nest and a plate hotel. The bead mover device may include a housing, a magnetic head assembly having an array of downwardly extending elongated magnets, and a leadscrew located within the housing and configured to move the magnetic head assembly along a vertical axis. The working nest may be located on the frontside of the housing below the magnetic head assembly, and the plate hotel may be located on the backside of the housing and configured to hold a plurality of fixtures when they are not in use on the working nest. The method may further include placing a sample microplate containing samples to be purified in the working nest, lowering the magnet array by computer command into the sample microplate such that magnetic beads are captured by the magnet array, and raising the magnet array by computer command such that the captured magnetic beads with nucleotides upon their surfaces are removed from the sample microplate. The sample microplate may then be moved from the working nest and placed in the plate hotel, and a first wash microplate may be moved from the plate motel and placed in the working nest. The method may further include lowering the magnet array by computer command into the first wash microplate such that magnetic beads enter wells of the first wash microplate, and raising the magnet array by computer command such that the captured magnetic beads with nucleotides upon their surfaces are removed from the first wash microplate. The first wash microplate may be removed from the working nest, and an elution microplate may be moved from the plate motel and placed in the working nest. The method may further include lowering the magnet array by computer command into the elution microplate such that magnetic beads enter wells of the elution microplate, raising the magnet array by computer command such that the captured magnetic beads with nucleotides upon their surfaces are removed from the elution microplate, and removing the elution microplate from the working nest.

In some embodiments, the above method further includes moving a second wash microplate from the plate motel and placing it in the working nest, lowering the magnet array by computer command into the second wash microplate such that magnetic beads enter wells of the second wash microplate, raising the magnet array by computer command such that the captured magnetic beads with nucleotides upon their surfaces are removed from the second wash microplate, and removing the second wash microplate from the working nest.

In some embodiments, the above method further includes moving a third wash microplate from the plate motel and placing it in the working nest, lowering the magnet array by computer command into the third wash microplate such that magnetic beads enter wells of the third wash microplate, raising the magnet array by computer command such that the captured magnetic beads with nucleotides upon their surfaces are removed from the third wash microplate, and removing the third wash microplate from the working nest. Each of the first, second and third wash microplates may be returned to the plate hotel after use.

In some embodiments, the plate hotel is provided with a series of shelves having an orientation that is parallel to an orientation of the working nest.

In some embodiments, the above method further includes placing a cover plate into a cover plate loading manifold, placing the cover plate loading manifold into the plate hotel, moving the cover plate loading manifold with the cover plate from the plate hotel to the working nest, lowering the magnet array by computer command into the cover plate such that the cover plate is retained on the magnet array, raising the magnet array with the retained cover plate by computer command, and moving the cover plate loading manifold without the cover plate from the working nest to the plate hotel.

In some embodiments, the above method further includes placing a cover plate ejection manifold into the plate hotel, moving the cover plate ejection manifold from the plate hotel to the working nest, lowering the magnet array by computer command into the cover plate ejection manifold, ejecting the cover plate from the magnet array into the cover plate ejection manifold, raising the magnet array by computer command, and moving the cover plate ejection manifold with the ejected cover plate from the working nest to the plate hotel.

### BRIEF DESCRIPTION OF THE DRAWINGS

FIG. 1, consisting of FIGS. 1a through 1c, is a diagrammatic representation of a generic prior art apparatus for magnetic bead nucleotide purification, having a z-axis slide 1, an array of magnets 3 and a well plate 5.

FIG. 2, consisting of FIGS. 2a though 2d, is a diagrammatic representation showing certain steps of the prior art magnetic bead nucleotide purification machine process, involving magnetic beads 2, well or container 111 and magnetic bar cover 121.

FIG. 3, consisting of FIGS. 3a through 3d, is a diagrammatic representation of an automated device for the modified magnetic bead nucleotide purification machine method in accordance with the present disclosure.

FIG. 4 is a flow chart particularly showing the computer software control of the magnetic head mover of the device in accordance with the present disclosure previously seen in FIG. 3.

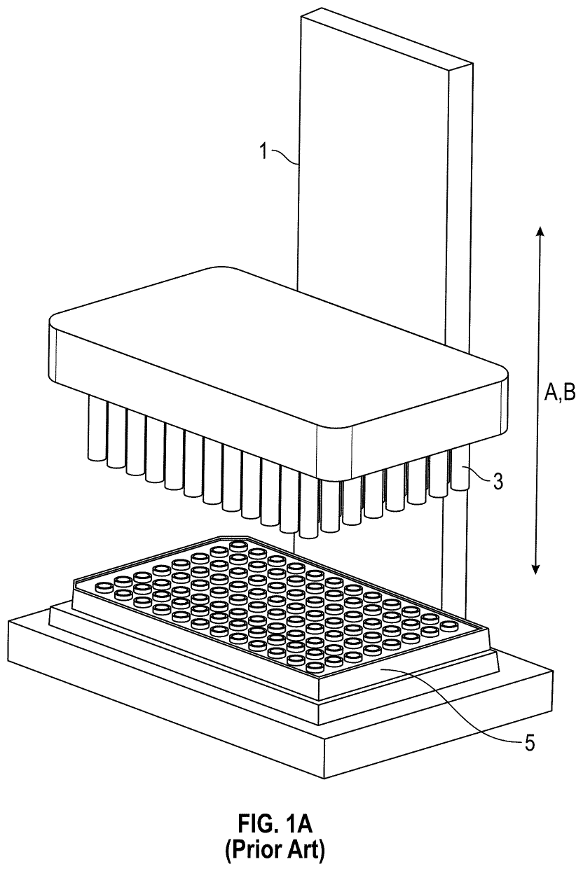

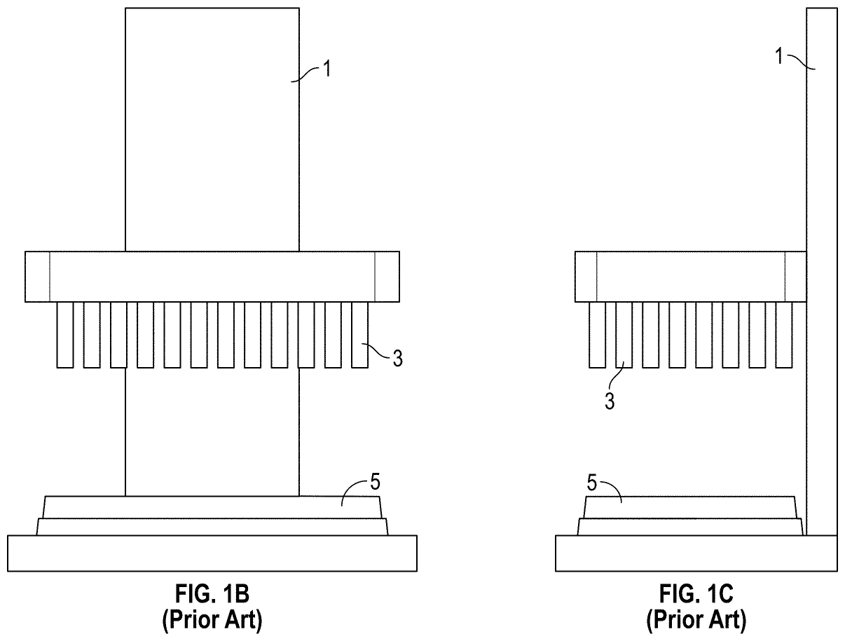

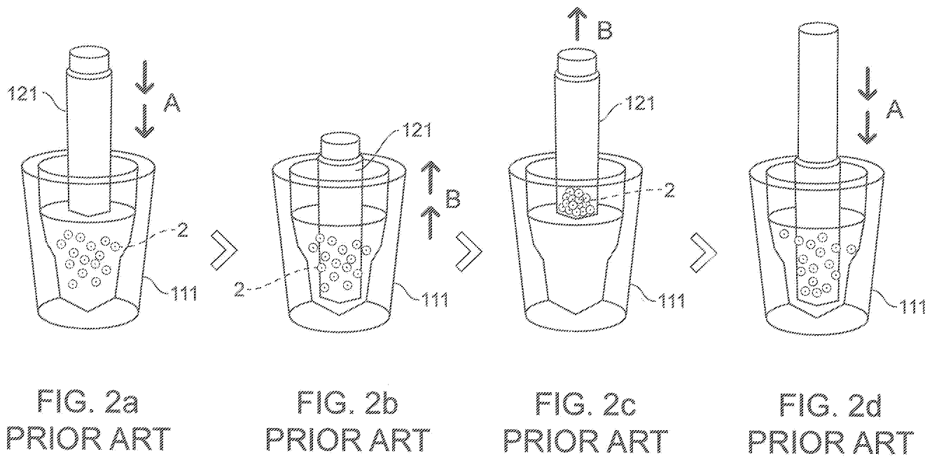

### DETAILED DESCRIPTION

### The General System and Device of the Present Disclosure

Purification processing in accordance with the present disclosure is often performed in the wells of an industry standard ninety-six (96) well microplate. Microplates containing a different number of sample wells may be used. As is conventional, samples and reagents are added to the wells of the plates prior to their use in the magnetic bead purification process.

An exemplary magnetic bead mover system in accordance with the present disclosure includes a bead mover device 10, a cover plate loader manifold 12, a cover plate ejection manifold 14, and at least one 1×1 ml 96-well microplate 16 with a 500 ul sample to be purified. Magnetic beads are added to this plate before the purification procedure. This microplate may also be incubated on a plate shaker to ensure nucleotide binding to the surfaces of the beads.

Also included are typically three shallow 96-well microplate plates 18. Each well of each plate 18 normally holds 200 ul ethanol or another appropriate wash solution.

Finally used in this exemplary embodiment is one 96-well microplate plate 20 (not shown) with 50 ul elution solution.

### The General Machine Method of Use of the System and Device of the Present Disclosure

First, in use of the present device, each microplate 16, 18, 20 is normally manually loaded onto the tray or plate hotel 22 attached to the bead mover 10. A cover plate 24 and a loading manifold 12 will be moved to the working nest 26.

The magnetic head 28 is then lowered so that the cover plate 24 clicks into place over the bottom of the magnetic rods of head 28.

The loading manifold 12 is then returned to its nest in the plate hotel 22.

Next, the magnetic head 28 and cover plate 24 move up and out of the way and the sample plate 16 is moved to the working nest 26.

The magnetic head 28 moves down into the sample plate 16. The bottom of the cover plate 24 normally rests 0.5 to 1 mm from the bottom of the sample plate 16. The magnetic head 28 rests in the sample plate 16 for a duration preset by the user.

The magnetic head 28 and cover plate 24 are then moved up and out of the sample plate 16 slowly to prevent dripping. The sample plate 16 is normally removed and returned to its original nest in the plate hotel 22.

Next in the exemplary method of the present disclosure comes the step of washing. A first wash plate 18 been loaded onto the working nest 26. The magnetic head 28 is lowered into the washing plate 18.

The magnetic head 28 with cover plate 24 is dipped into the wash plate 18 a number of times that is set by the user. Normally three times is the default number of dippings, but this may be easily changed in the control software.

This washing sequence is normally repeated for each of three wash plates 18. The last wash plate 18 will be returned to its original nest in plate hotel 22.

Next to last in this exemplary method of the present disclosure comes the step of elution. An elution plate 20 (not shown) will be removed from its nest and moved to the working nest.

The magnetic head 28 is lowered into the elution solution and rests there for a time preset by the user. The default time is thirty seconds (30 s), although this may be readily changed in the control software.

The elution solution volume will often be fifty microliters (50 ul) or less, so in some embodiments the cover plate 24 lowers to the very bottom of the elution plate 20 such that the magnetic beads are covered by the elution solution.

After elution, the elution plate 20 is returned to its original nest. The purified sample will be in this plate.

Final steps in this exemplary method include housekeeping steps. The cover plate 24 is ejected as follows.

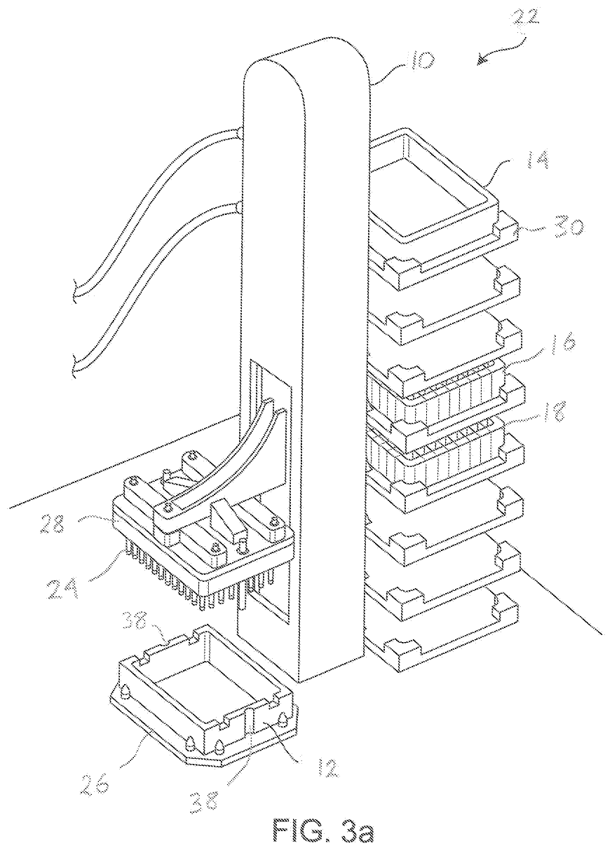

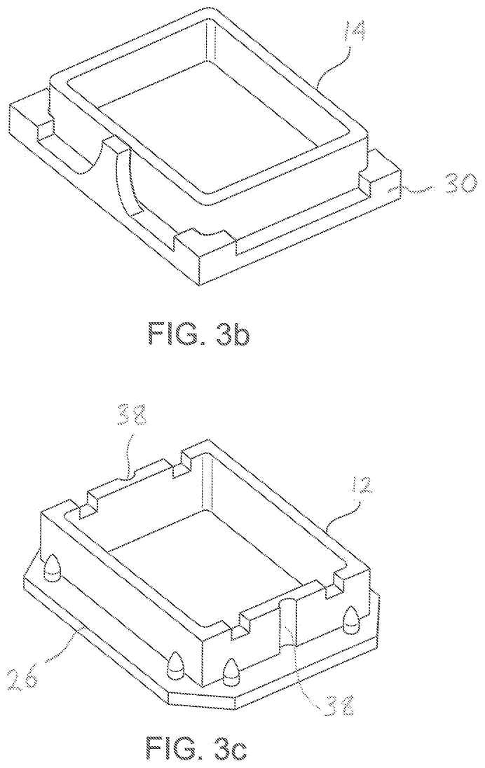

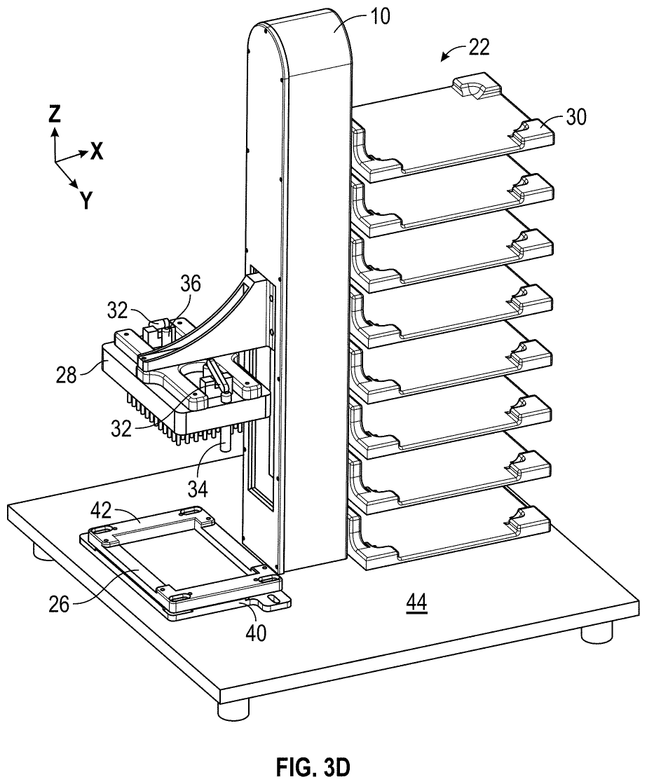

The ejection manifold 14 is moved to the working nest 26. The magnetic head 28 is moved down, and the cover plate 24 is ejected. As best seen in FIG. 3d, magnetic head 28 may be provided with a pair of toggles 32 on opposite sides of head 28. Each toggle may be provided with a downwardly projecting outer pin 34 and a downwardly projecting inner pin 36. Each toggle may be spring-loaded such that the outer pin 34 is biased towards a downward position. When magnetic head 28 is lowered, outer pins contact the side walls of ejection manifold 14 (shown in FIG. 3b). As outer pins 34 move upwardly relative to magnetic head 28, the toggles 32 urge inner pins 36 downward, thereby ejecting cover plate 24 onto ejection manifold 14. The ejected cover plate 24 and the manifold 14 are returned to their original nest with the magnetic beads now located in the ejection manifold with the cover plate 24. Loading manifold 12 (shown in FIG. 3c) and other fixtures and/or plates may be provided with cutouts 38 such that outer pins 34 do not contact the fixture and eject the cover plate 24 prematurely.

In some embodiments, tray hotel 22 is formed by eight shelves 30 rigidly attached to the backside of bead mover 10. In other embodiments, there may be more or fewer shelves 30. For example, tray hotel 22 may include six shelves such that the seven previously described plates and fixtures may be accommodated on shelves 30 and working nest 26. Shelves 30 may be oriented in the same direction as working nest 26, as shown in FIG. 3a, or they may be oriented perpendicularly, as shown in FIG. 3d. The arrangement of working nest 26, bead mover 10 and tray hotel 22, particularly as shown in FIG. 3a, provides a compact, all-inclusive system that facilitates efficient transfers of well plates, fixtures and other items between working nest 26 when they are in use and tray hotel 22 when these items are not in use. These transfers can be made manually by a user standing or sitting at a lab bench, automatically by an automated transfer system, or a combination of the two. In other words, shelves 30 of tray hotel 22 are out of the way of working nest 26, but still within reach of an operator seated in front of the system 10. Additionally, system 10 can hold a large number of well plates and fixtures without taking up much lab bench space. When the arrangement of FIG. 3a is used, trays and fixtures need not be rotated when being moved between working nest 26 and shelves 30. In some embodiments, such as the one shown in FIG. 3d, base 44 is no larger than 15 inches wide and 15 inches deep. In this embodiment, the bead mover device, the working nest and the plate hotel all fit within a vertical projection of the base so that multiple workstations may be placed side by side on a lab bench. In other embodiments, such as the one shown in FIG. 3a, base 44 is no larger than 12 inches wide and 12 inches deep. In this embodiment, the bead mover device, the working nest and the plate hotel all fit within an even more compact vertical projection of the base.

As best seen in FIG. 3d, working nest 26 may be formed from a lower frame 40 and an upper frame 42. Lower frame 40 may be provided with elongated slots that allow it to be adjusted and secured in an x-direction relative to base 44 and bead mover 10. Upper frame 42 may also be provided with elongated slots, allowing it to be moved independently from lower frame 40, and adjusted and secured in a y-direction. This arrangement allows working nest 26 (and therefore the plates and fixtures that are alternately placed upon it) to be accurately registered relative to the downwardly projected magnets of head 28.

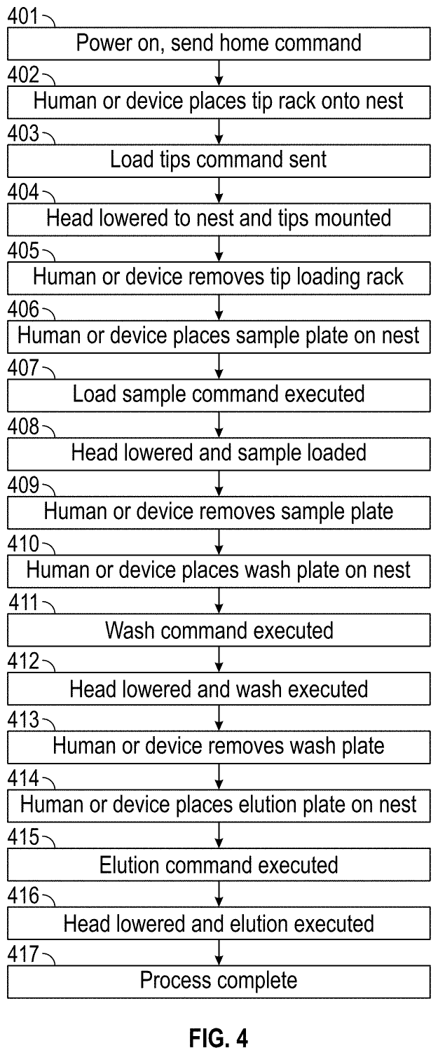

Considering now FIG. 4, a flow chart for the machine performance of another exemplary process of the present disclosure is shown.

The machine is powered on in block 401, clean tips for ninety-six magnets are positioned in a tip rack in block 402, and by command in block 403 a Z-axis motor moveable head is cycled downward to mount all the tips from the loading rack in block 404, and, with the magnetic head drawn back upwards with tips now mounted in block 405, the automated machine device is ready to enter into the process of “magnetic bead purification”.

Commencing the process, a microplate with samples to be purified is placed under the magnetic head in a work center column called the “nest” in block 406. By computer command in block 407 the magnetic head is lowered, the magnetic beads are captured, and the captured magnetic beads are removed from each microplate well in block 408. The magnetic beads (with nucleotides upon their surfaces) now being held at the tips of the down-hanging magnets of the raised magnetic head, the sample microplate is now removed in block 410.

Continuing in the process of magnetic bead purification, a wash step (which may be repeated) and an elution step are shown in the flowchart of FIG. 4. A wash plate is placed into the nest—being that site on the machine where is located the vertically, “Z” axis, moveable magnetic head—in block 410. This placement, and a subsequent withdrawal of the wash plate in block 413, may be done by human or by machine. All the automated mechanical motions flowcharted in FIG. 4 are straightforward and may readily be implemented by a modern computer-controlled machine designer. The magnetically held magnetic beads bearing the samples being lowered into the wash microplate by computer command, a wash is executed in block 412. This wash step from block 411 to block 413 may be repeated any desired number of times. For most samples undergoing magnetic bead purification, three wash cycles are commonly used.

Finally, in FIG. 4, an elution step using an elution microplate is performed. This elution microplate is placed—again by human or by machine—on the nest in block 414 and then, by computer command in block 415, the magnetic head and its held magnetic beads are lowed into the elution microplate in block 416, completing the purification process where the eluted nucleotides are present in block 417,

Throughout the flow chart of FIG. 4 it will be recognized that items are being moved in space relative to each other, and all motions are relative and should be so interpreted in the claims that follow.

Additionally, it is contemplated that automated magnetic bead purification in accordance with the methods and the machine devices of the present disclosure is extendible to new, and/or to modified and/or enhanced and/or repeated steps, and new steps, without deviation from the spirit of the disclosure.

In accordance with the general concept of the concentration, purification and/or isolation of nucleotides including DNA/RNA, proteins and cells by process of magnetic bead separation, modifications and additions to the steps, and to the system and device, just described will suggest themselves to a practitioner not only of biological laboratory processes, but also to a practitioner of computer-controlled machine design. Therefore, the scope of the present disclosure should be determined by the claims that will follow, only, and not solely in accordance with those embodiments within which the disclosure has been described.

What is claims is:

1. An automated workstation configured for magnetic bead nucleotide purification, the workstation comprising:
a bead mover device, the bead mover device comprising;
a housing;
a magnetic head assembly having an array of downwardly extending elongated magnets; and
a leadscrew located within the housing and configured to move the magnetic head assembly along a vertical axis;
a working nest located on a frontside of the housing below the magnetic head assembly; and
a plate hotel located on a backside of the housing and configured to hold a plurality of fixtures when not in use on the working nest.

2. The workstation of claim 1, further comprising a computer processor configured to control the leadscrew in moving the magnetic head along the vertical axis.

3. The workstation of claim 1, wherein the plate hotel comprises a plurality of shelves attached to the backside of the bead mover device housing.

4. The workstation of claim 3, wherein the plate hotel comprises at least 6 shelves.

5. The workstation of claim 3, wherein the plate hotel comprises at least 8 shelves.

6. The workstation of claim 3, wherein the shelves have an orientation that is parallel to an orientation of the working nest.

7. The workstation of claim 3, wherein the shelves have an orientation that is perpendicular to an orientation of the working nest.

8. The workstation of claim 1, wherein the array of downwardly extending elongated magnets is configured to be removably covered by cover plate, wherein the cover plate is an industry standard ninety-six well microplate.

9. The workstation of claim 8, wherein the workstation further comprises a cover plate loading manifold configured to removably receive the cover plate, be temporarily stored in the plate hotel, and transferred to the working nest to apply the cover plate to the array of magnets before use.

10. The workstation of claim 8, wherein the workstation further comprises a cover plate ejection manifold configured to be temporarily stored in the plate hotel, transferred to the working nest, and receive the cover plate from the array of magnets after use.

11. The workstation of claim 10, wherein the magnetic head assembly is provided with a pair of toggles on opposite sides of the magnetic head assembly configured to automatically eject the cover plate when the magnetic head assembly is lowered by the leadscrew onto the cover plate ejection manifold.

12. The workstation of claim 1, wherein the workstation further comprises a base to which a bottom end of the bead mover device housing is attached, wherein the working nest comprises a lower frame provided with elongated slots that allow it to be adjusted and secured in an x-direction relative to the base and the bead mover device, and wherein the working nest comprises an upper frame also provided with elongated slots allowing it to be moved independently from the lower frame, adjusted and secured in a y-direction, thereby allowing the working nest and therefore plates and fixtures that are alternately placed upon the working nest to be accurately registered relative to the array of downwardly extending magnets.

13. The workstation of claim 1, wherein the workstation further comprises a base to which a bottom end of the bead mover device housing is attached, wherein the base is no larger than 15 inches wide and 15 inches deep, and wherein the bead mover device, the working nest and the plate hotel all fit within a vertical projection of the base.

14. A method of purifying biological material using magnetic beads, the method comprising:
providing an automated workstation, the workstation comprising:
a bead mover device, the bead mover device comprising;
a housing;
a magnetic head assembly having an array of downwardly extending elongated magnets; and
a leadscrew located within the housing and configured to move the magnetic head assembly along a vertical axis;
a working nest located on a frontside of the housing below the magnetic head assembly; and
a plate hotel located on a backside of the housing and configured to hold a plurality of fixtures when not in use on the working nest;
placing a sample microplate containing samples to be purified in the working nest;
lowering the magnet array by computer command into the sample microplate such that magnetic beads are captured by the magnet array;
raising the magnet array by computer command such that the captured magnetic beads with nucleotides upon their surfaces are removed from the sample microplate;
moving the sample microplate from the working nest and placing it in the plate hotel;
moving a first wash microplate from the plate motel and placing it in the working nest;
lowering the magnet array by computer command into the first wash microplate such that magnetic beads enter wells of the first wash microplate;
raising the magnet array by computer command such that the captured magnetic beads with nucleotides upon their surfaces are removed from the first wash microplate;
removing the first wash microplate from the working nest;
moving an elution microplate from the plate motel and placing it in the working nest;
lowering the magnet array by computer command into the elution microplate such that magnetic beads enter wells of the elution microplate;
raising the magnet array by computer command such that the captured magnetic beads with nucleotides upon their surfaces are removed from the elution microplate; and
removing the elution microplate from the working nest.

15. The method of claim 14, further comprising:
moving a second wash microplate from the plate motel and placing it in the working nest;
lowering the magnet array by computer command into the second wash microplate such that magnetic beads enter wells of the second wash microplate;
raising the magnet array by computer command such that the captured magnetic beads with nucleotides upon their surfaces are removed from the second wash microplate;
removing the second wash microplate from the working nest.

16. The method of claim 15, further comprising:
moving a third wash microplate from the plate motel and placing it in the working nest;
lowering the magnet array by computer command into the third wash microplate such that magnetic beads enter wells of the third wash microplate;
raising the magnet array by computer command such that the captured magnetic beads with nucleotides upon their surfaces are removed from the third wash microplate;
removing the third wash microplate from the working nest.

17. The method of claim 16, wherein each of the first, the second and the third wash microplates are returned to the plate hotel after use.

18. The method of claim 14, wherein the plate hotel is provided with a series of shelves having an orientation that is parallel to an orientation of the working nest.

19. The method of claim 14, further comprising:
placing a cover plate into a cover plate loading manifold;
placing the cover plate loading manifold into the plate hotel;
moving the cover plate loading manifold with the cover plate from the plate hotel to the working nest;
lowering the magnet array by computer command into the cover plate such that the cover plate is retained on the magnet array;
raising the magnet array with the retained cover plate by computer command; and
moving the cover plate loading manifold without the cover plate from the working nest to the plate hotel.

20. The method of claim 14, further comprising:
placing a cover plate ejection manifold into the plate hotel;
moving the cover plate ejection manifold from the plate hotel to the working nest;
lowering the magnet array by computer command into the cover plate ejection manifold;
ejecting the cover plate from the magnet array into the cover plate ejection manifold;
raising the magnet array by computer command; and
moving the cover plate ejection manifold with the ejected cover plate from the working nest to the plate hotel.
